(19) United States
(12) Patent Application Publication
(10) Pub. No.: US 2018/0333347 A1
(43) Pub. Date: Nov. 22, 2018

(54) PHOTOCHEMICALLY STABLE, NON-LEACHING SUNSCREENS FROM EPOXIDE-BASED SILANE COUPLING AGENTS AND UV ABSORBING CHROMOPHORES

(71) Applicant: ARIZONA BOARD OF REGENTS ON BEHALF OF THE UNIVERSITY OF ARIZONA, Tucson, AZ (US)

(72) Inventors: Douglas A. Loy, Tucson, AZ (US); Peter McFadden, Tucson, AZ (US)

(21) Appl. No.: 15/980,617

(22) Filed: May 15, 2018

Related U.S. Application Data
(60) Provisional application No. 62/506,972, filed on May 16, 2017.

(51) Int. Cl. A61K 8/895; A61Q 17/04; C08G 77/20

(52) U.S. Cl. CPC A61K 8/895; C08G 77/20; A61Q 17/04

(57) ABSTRACT

UV absorbing monomers having one or more alkoxysilyl functional groups are produced through epoxide reaction chemistry. Sol-gel or emulsion polymerization of said UV absorbing monomers by themselves, or with alkoxysilane comonomers, affords nanoparticle sunscreens with reduced chemical exposure and improved photostability. Minimized leaching and decreased levels of photo-degradation were achieved with covalent incorporation to ensure isolation of the sunscreen chromophores and any photo-products from the skin.

### CROSS REFERENCE

This application claims priority to U.S. Patent Application No. 62/506,972, filed May 16, 2017, the specifications of which are incorporated herein in their entirety by reference.

### FIELD OF THE INVENTION

The present invention relates to sunscreens, in particular, to sunscreens based on nanoparticles of epoxide-based silane coupling agents and naturally occurring UV absorbing chromophores.

### BACKGROUND OF THE INVENTION

UV radiation (100-400 nm) represents ˜10% of electromagnetic radiation (sunlight) that reaches the Earth's surface, and can be separated into three main types: UVC (100-290 nm), which has the shortest wavelength and highest energy; UVB (290-320 nm); and UVA (320-400 nm), which has the longest wavelength and lowest energy. UVA is further divided into UVA-I (340-400 nm) and UVA-II (320-340 nm). In small doses, UV radiation can be beneficial for vitamin D production or as therapeutic treatment for skin disorders, but too much exposure can cause several harmful photo-biological effects such as erythema (sunburn); accelerated skin ageing resulting in a variety of visible effects such as pronounced deep furrows, sagging, wrinkles, uneven pigmentation, dryness, and a leathery appearance; and most alarming, skin cancer. In the case of the latter, exposure accounts for the development of approximately 90% of all non-melanoma carcinomas according to the Environmental Protection Agency (EPA), with an estimated 3.5 million+ new cases of basal and squamous cell carcinoma diagnosed in the United States each year.

UVB has traditionally been thought to be the most harmful radiation to skin, and the prevalence of UVB absorbing active ingredients in commercial sunscreens has reflected this belief. Recent studies, however, have detailed the harmful effects of UVA exposure as well, for sufficient doses of UVA, particularly UVA-II, can in fact induce sunburn. Further still, elastosis can be induced by both UVB and UVA radiation. Due to the deeper penetration depth of UVA in skin, increased absorption of UVA in persons protected with sunscreens that only filter UVB and extended exposure to the sun without the warning of sunburn, UVA has been found to be the major contributor of accelerated skin aging.

The use of sunscreens, which contain active ingredients that block UV, is recommended to mitigate UV exposure. Organic absorber sunscreens provide protection by preventing the penetration of UV radiation into skin via absorption of high energy UV. Examples of said compounds include, but are not limited to, salicylates, cinnamates, benzophenone, p-aminobenzoic acid and analogues (PABAs), dibenzoylmethanes, and camphor derivatives. While these examples are successful at absorbing UV radiation, they are limited in safety and effectiveness over time due to UV-induced decomposition and photo-toxicity, as well as photo-allergenic effects. For example, salicylates are UVB absorbing sunscreens that can cause allergic reactions and skin inflammation. Sunscreen compounds are even known to systemically absorb into the body after topical application, leading to concerns about their estrogenicity. Thus, there exists a need for sunscreens that are photochemically stable in the presence of UV and non-leeching to ensure their continual use.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

It is an objective of the present invention to provide for sunscreen compositions that have improved UV stability, as well as minimal sunscreen leaching, as specified in the independent claims. Embodiments of the invention are given in the dependent claims. Embodiments of the present invention can be freely combined with each other if they are not mutually exclusive.

In one embodiment, nanoparticle sunscreens can be prepared by polymerizing UV absorbing monomers bearing one or more trialkoxysilyl groups or two or more methyldialkoxysilyl groups with themselves, or with tetraethoxysilane or organotrialkoxysilane comonomers, via sol-gel or emulsion polymerizations. The sub-micron sized particles can contain up to 100 mol % of the UV absorbing chromophore, thereby making them ideally suited as sunscreens without the potentially hazardous effects of exposure to liquid organic sunscreens. Furthermore, integration into solid state matrices increases the photostability of the chromophores substantially over that of the liquid sunscreens used in traditional sunscreens.

A unique and inventive technical feature of the present invention is the sunscreen particles prepared by reacting silane coupling agents bearing epoxy groups with UV absorbing chromophore bearing nucleophilic hydroxyl, amine, thiol or carboxylic acid groups. This allows UV absorbing chromophores to be easily integrated into monomers used in the formation of the sunscreen nanoparticles. Without wishing to limit the invention to a particular theory or mechanism, the covalent bonding of the silane coupling agents to the UV absorbing chromophore is effective for preventing leeching of the UV absorbing chromophore from the sunscreen particle. Moreover, the sunscreen particle may be resistant to photo-degradation. Thus, the present invention can advantageously provide safer and more effective sun care products that reduce exposure to chemicals and have improved stability. None of the presently known prior references or work has the unique inventive technical feature of the present invention

### DESCRIPTION OF PREFERRED EMBODIMENTS

As used herein, a UV chromophore can be a molecule or moiety that absorbs UV light. In preferred embodiments, the UV chromophore may have nucleophilic hydroxyl, amine, thiol, or carboxylic acid groups. As known to one of ordinary skill in the art, the term “nucleophile” refers to a molecule or ion that donates a pair of electrons to form a new covalent bond. A nucleophile is also known as a Lewis base. As used herein, photostability refers to the characteristic of resistance to degredation or oxidation due to solar, UV, or visable light.

According to one embodiment, the present invention features a composition for producing a UV absorbing sunscreen monomer having one or more alkoxysilyl functional groups. The composition may comprise a UV absorbing compound, wherein the UV absorbing compound contains at least one nucleophilic functional group, and one or more silane coupling agents having at least one epoxide group. Without wishing to limit the invention to a particular theory or mechanism, the nucleophilic functional group may be configured to react with the epoxide group to form a covalent bond, thereby producing the sunscreen monomer, wherein the covalent bond is effective for enhancing photostability of the sunscreen monomer and preventing leeching of the UV absorbing compound from the sunscreen monomer.

According to another embodiment, the present invention features a method for producing a photochemically stable, non-leeching sunscreen monomer for use in a sunscreen formulation. In some embodiments, the method may comprise providing the UV absorbing compound, where the UV absorbing compound preferably contains at least one nucleophilic functional group, providing one or more silane coupling agents having at least one epoxide group, and reacting the UV absorbing compound with the one or more silane coupling agents such that the nucleophilic functional group of the UV absorbing compound covalently binds to the epoxide group of the silane coupling agent, thereby forming the photochemically stable sunscreen monomer. Without wishing to limit the invention to a particular theory or mechanism, the covalent bonding of the nucleophilic functional group to the epoxide group may be effective for enhancing photostability of the sunscreen monomer and preventing leeching of the UV absorbing compound from the sunscreen monomer.

In some embodiments, the UV absorbing compound is a chromophore that can absorb UV radiation. In other embodiments, the nucleophilic functional group of the UV absorbing compound is a hydroxyl moiety, an amine moiety, a thiol moiety, or a carboxylic acid moiety.

Non-limiting examples of UV chromophores having nucleophilic hydroxyl groups include curcumin, resveratrol, catechin, ellagic acid, usnic acid, hematoxylin/hematein, kermesic acid, carminic acid, caffeic acid, ferulic acid, 3,5-dicaffeoylquininic acid, 5,6,7-trihydroxy-2-phenyl-4H-1-benzopyran-4-one, salvianolic acid, 2′,4,4′-trihydroxychalcone, 3,5,7,3′,4′,5′-hexahydroxyflavone, 3,5,7,3′,4′,5′-hexahydroxyflavone, 5,7-dihydroxy-2-phenylchromen-4-one, 5,7-dihydroxy-3-(4-methoxyphenyl)-4H-1-benzopyran-4-one, 5,7,4′-trihydroxyflavonol, 5,7-dihydroxy-2-(4-hydroxyphenyl)-4H-chromen-4-one, 5,7-dihydroxy-3-(4-hydroxyphenyl)-4H-chromen-4-one, 3′,4′,5,7-tetrahydroxyflavone, and bis-ethylhexyloxyphenol methoxyphenyl triazine.

Examples of UV chromophores having nucleophilic amine groups (e.g. NH or NH₂) include, but are not limited to, 1,3-phenylenebis((3-aminophenyl)methanone), 4,4′-diamino-2,2′-stilbenedisulfonic acid, and 2-(4-aminophenyl)-1H-benzimidazole-5-amine).

Non-limiting examples of UV chromophores having nucleophilic thiol groups include thiolated avobenzones such as 1,3-bis(4-mercaptophenyl)propane-1,3-dione, oxybenzones such as bis(4-mercaptophenyl)methanone, thiolated coumarins such as 7-mercaptocoumarin, cinnamates such as 2-ethylhexyl (E)-3-(4-mercaptophenyl)-acrylate, salicylates such as sec-butyl 2-mercaptobenzoate, anthranilate such as 2-mercaptoethyl 2-(methylamino)benzoates, bisoctrizoles such as 6,6′-methylenebis(2-(2H-benzo-[d][1,2,3]triazol-2-yl)-4-(2,4,4-trimethylpentan-2-yl)benzenethiol), and bemotrizinols such as 6,6′-(6-(4-mercaptophenyl)-1,3,5-triazine-2,4-diyl)bis(3-((2-ethylhexyl)oxy)phenol).

Examples of UV chromophores having nucleophilic carboxylic acid groups include, but are not limited to, flavonoids such as coumarin-3-carboxylic acid and fumaric acid, carboxylated benzophenones such as 4,4′-carbonyldibenzoic acid and 3-(2-(2-(2-carboxyethyl)-6-hydroxybenzoyl)phenyl)propanoic acid, avobenzones such as 3-(2-(3-(4-(tert butyl)-2-(2-carboxyethyl)phenyl)-3-oxopropanoyl)-5-hydroxyphenyl)-propanoic acid, anthranilates such as 3-(2-(sec-butoxycarbonyl)-3-(methylamino)-phenyl)propanoic acid, bisoctriazoles such as 3,3′-((methylenebis(2-hydroxy-5-(2,4,4-trimethylpentan-2-yl)-3,1-phenylene))bis(2H-benzo[d][1,2,3]triazole-2,4-diyl))dipropionic acid, and bemotrizinols such as 4-(4,6-bis(4-((2-ethylhexyl)oxy)-2-hydroxyphenyl)-1,3,5-triazin-2-yl)benzoic acid.

In some embodiments, non-limiting examples of the silane coupling agent include glycidyloxypropyltrimethoxysilane, glycidyloxypropyltriethoxysilane, glycidyloxypropyldimethoxymethylsilane, glycidyloxypropyldiethoxymethylsilane, (2-(7-oxabicyclo-[4.1.0]heptan-3-yl)ethyl)trimethoxysilane, (2-(7-oxabicydo[4.1.0]heptan-3-yl)ethyl)-triethoxysilane, (2-(7-oxabicyclo[4.1.0]heptan-3-yl)ethyl)dimethoxymethylsilane, (2-(7-oxabicyclo[4.1.0]heptan-3-yl)ethyl)diethoxymethylsilane, trimethoxy(2-(oxiran-2-yl)ethyl)-silane, triethoxy(2-(oxiran-2-yl)ethyl)silane, methyldimethoxy(2-(oxiran-2-yl)ethyl)silane, methyldiethoxy(2-(oxiran-2-yl)ethyl)silane, trimethoxy(oxiran-2-ylmethyl)silane, triethoxy-(oxiran-2-ylmethyl)silane), methyldimethoxy(oxiran-2-ylmethyl)silane, and methyl-diethoxy(oxiran-2-ylmethyl)silane.

A non-limiting example of sunscreen monomer of the present invention is illustrated in Scheme 1.

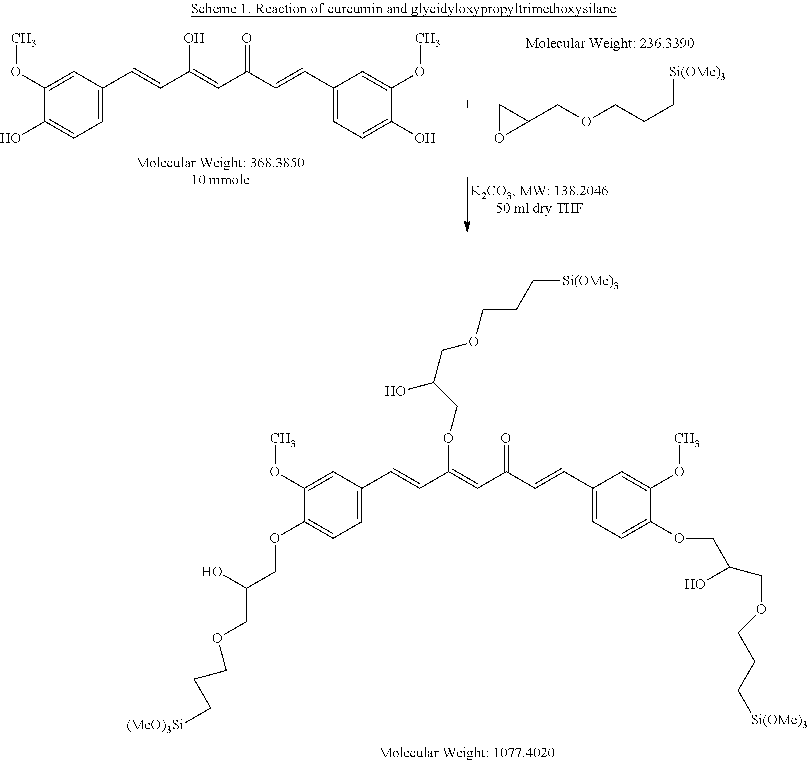

According to other embodiments, the methods of the present invention may further comprise polymerizing a plurality of the sunscreen monomers with each other to form sunscreen nanoparticles. The nanoparticles may be formed by polymerization of the alkoxysilyl groups. In an alternative embodiment, the methods of the present invention may further comprise polymerizing the plurality of the sunscreen monomers with silicate comonomers to form the sunscreen nanoparticles. In any case, these sunscreen nanoparticles can be a polymer matrix having the UV absorbing compound embedded therein. In other embodiments, the plurality of the sunscreen monomers is polymerized by themselves or with the silicate comonomers by sol gel polymerization or emulsion polymerization.

In one embodiment, the sunscreen nanoparticles may be irregularly spherical in shape. For instance, each sunscreen particle can have a mean diameter of about 50 to 750 nm.

In some embodiments, the silicate comonomers may be tetraalkoxysilane comonomers, sodium silicate comonomers, or organotrialkoxysilane comonomers. For example, the tetraalkoxysilane comonomers may be tetramethoxysilane comonomers or tetraethoxysilane comonomers.

In another embodiment, the organotrialkoxysilanes can be according to the formula: (R′O)₃Si—R, where R′ can be a methyl or ethyl, and R can be a methyl, ethyl, propyl, butyl, phenyl, vinyl, allyl, chloromethyl, 2-chloroethyl, or hydroxymethyl. For instance, the organotrialkoxysilane can be a methyltriethoxysilane. In another embodiment, the organotrialkoxysilanes can be according to the formula: (R′O)₃Si—R—Si(OR′)₃, where R′ can be a methyl or ethyl, and R can be a 1,4-phenylene, 1,3-phenylene, 4,4′-biphenylene, methylene, 1,2-ethylene, 1,2-ethenylene, 1,3-propylene, 1,6-hexylene, 1,8-octylene, 1,10-decylene. It is to be understood that any appropriate organotrialkoxysilanes can be used in accordance with the present invention, and that the present invention is not limited to the examples of the organotrialkoxysilanes described herein.

According to some embodiments, the present invention may feature a sunscreen composition comprising a plurality of sunscreen nanoparticles. Each of the sunscreen nanoparticles may comprise a plurality of any one of the sunscreen monomers described herein. In one embodiment, the plurality of sunscreen monomers may be polymerized with each other to form the sunscreen nanoparticles. In another embodiment, each nanoparticle may further comprise silicate comonomers. The sunscreen monomers may be polymerized with the silicate comonomers to form the sunscreen nanoparticles. Preferably, the technique of polymerization is sol-gel polymerization or emulsion polymerization.

In yet another embodiment, the present invention features a sunscreen formulation comprising any of the sunscreen compositions described herein, a cosmeceutically-acceptable sunscreen carrier comprising at least one solubilizer and at least one cosmetic adjuvant selected from the group consisting of preservatives, antifoams, perfumes, oils, waxes, propellants, dyes, pigments, film-forming and waterproofing agents, emulsifiers, surfactants, thickeners, binders, humectants, exfoliants and emollients. Preferably, the sunscreen composition is present in an amount effective to absorb UV radiation. In some embodiments, the sunscreen formulation is in a form suitable for topical application. Examples include, but are not limited to, creams, ointment, suspensions, powders, lotions, gels, solids, foams, emulsions, liquid dispersions, sprays and aerosols.

The sunscreen formulation may further comprise an anti-oxidant to aid in preventing or reducing erythema and boost SPF. Anti-oxidants can include, but are not limited to, natural polyphenols such as flavonoids (catechins), resveratrol, retinol, green tea extract, procyanidolic oligomers, vitamins C, vitamin E and other tocopherols, and natural oils such as rosemary, argan oil, and clove oil.

Solubilizers may be required to ensure adequate solubility of the sunscreen composition. Examples of sunscreen solubilizers, include, but are not limited to, solvents such as water, carrier oils such as castor oil, jojoba oil, cottonseed oil, peanut oil and sesame oil, vegetable oils, modified vegetable oils, alcohols, glycerin, butyloctyl salicylate, dimethyl capramide, diisobutyl adipate, etc.

In some embodiments, dispersing agents, emulsifiers or thickening agents in the sunscreen formulation can aid in applying a uniform layer of the sunscreen particle. Suitable dispersing agents for the sunscreen formulations include those useful for dispersing the hybrid organic/inorganic sunscreen particles in a water phase, an oil phase, or part of an emulsion.

Emulsifiers may be used to disperse the sunscreen composition of the formulation. Suitable emulsifiers include ethoxylated fatty acids, ethoxylated esters, ethoxylated ethers, ethoxylated alcohols, phosphated esters, polyoxyethylene fatty ether phosphates, fatty acid amides, acyl lactylates, soaps and mixtures thereof. Examples thereof include glycerol stearate, fatty alcohols such as stearyl alcohol, cetyl alcohol, cetearyl alcohol, cetearyl palmitate, lauryl myristate and isopropyl palmitate, dimethicone copolyol phosphate, lecithin, stearic acid, sugar emulsifiers such as sucrose ester and sorbitan ester, modified vegetable esters such as polyglyceryl-6 polyricinoleate, glyceryl oleate, phosphate esters such as castor oil, soy emulsifiers, vegetable and fermented gums, lanolin, botanical extracts, polyoxyethylene (8) stearate, myristyl ethoxy (3) myristate, polyoxyethylene (100) monostearate, lauric diethanolamide, stearic monoethanolamide, hydrogenated vegetable glycerides, sodium stearoyl-2-lactylate and calcium stearoyl-2-lactylate, soaps such as sodium stearate and triethanolamine stearate, lanolin and its derivatives and components such as acetylated lanolin, lanolin alcohols and lanolin fatty acids, etc.

Preservatives may be used to protect sunscreen formulation against microbial growth. Examples thereof include, but are not limited to glucose oxidase, lactoperoxidase, parabens such methyl, ethyl, propyl and butyl esters of hydroxybenzoic acid, EDTA, methylisothiazolinone, imidazolidinyl ureas, etc.

Thickening agents may be used to increase the viscosity of the sunscreen formulations. Suitable thickening agents include carbomers, acrylate/acrylonitrile copolymers, carboxyvinyl polymers, xanthan gum and combinations of these.

The sunscreen formulation may optionally contain an ingredient which enhances the waterproof properties and can form a polymeric film, such as acacia gum, rosin, esters, shellac, polyglyceryl-10 pentastearate, behenyl alcohol, lanolin, soluble collagen, polysaccharide based materials such as natural hydrocolloids, microcrystalline cellulose, modified cellulose, corn starch, dimethicone copolyol phosphate, diisostearoyl trimethyolpropane siloxysilicate, chitosan, dimethicone, polyethylene, PVP/Eicosene copolymer, adipic acids/diethylene glycol/glycerine crosspolymer, polyvinyl-pyrrolidone/vinylacetate, etc.

Skin conditioning agents can include humectants, exfoliants and emollients. Humectants are intended for moisturizing, reducing scaling and stimulating the removal of built scale from the skin. Examples include, but are not limited to, polyhydric alcohols such as propylene glycol, dipropylene glycol, polypropylene glycol, polyethylene glycol, sorbitol, 2-pyrrolidone-5-carboxylate, hydroxypropyl sorbitol, hexylene glycol, ethoxydiglycol 1,3-butylene glycol, 1,2,6-hexanetriol, glycerin, ethoxylated glycerin, alkoxylated glucose, hexanetriol, propoxylated glycerin and mixtures thereof. Exfoliants may be selected from alpha-hydroxy carboxylic acids, beta hydroxycarboxylic acids and salts of these acids.

Suitable emollients include those agents known for softening the skin or hair, which may be selected from hydrocarbons, fatty acids, fatty alcohols and esters. Waxes such as petrolatum, ceresin wax, carnauba wax, beeswax, and castor wax may be suitable emollients. For instance, petrolatum is a common hydrocarbon type of emollient conditioning agent. Other hydrocarbons that may be employed include alkyl benzoate, mineral oil, polyolefins such as polydecene, and paraffins, such as isohexadecane. Fatty acids and alcohols typically have from about 10 to 30 carbon atoms. Examples include, but are not limited to, myristic, isostearic, hydroxystearic, oleic, linoleic, ricinoleic, behenic and eruicic acids and alcohols. Ester emollients can include triglyceride esters, acetoglyceride esters, ethoxylated glycerides, alkyl esters of fatty acids, ether esters, polyhydric alcohol esters and wax esters. Additional emollients or hydrophobic agents include C₁₂ to C₁₅ alkyl benzoate, dioctyladipate, octyl stearate, octyldodecanol, hexyl laurate, octyldodecyl neopentanoate, cyclomethicone, dicapryl ether, dimethicone, phenyl trimethicone, isopropyl myristate, capriylic/capric glycerides, propylene glycol dicaprylate/dicaprate and decyl oleate.

In alternative embodiments, the sunscreen formulations may further contain inorganic sunscreen agents such as titanium dioxide and zinc oxide. In still other embodiments, the sunscreen formulation may further comprise plant extracts such as aloe vera, witch hazel, and cucumber. Further embodiments of the sunscreen formulation can include anti-aging and skin nutrifying ingredients such as retinoids, hydroquinone, alpha hydroxy acids, hyaluronic acids, vitamins A, B, C, and E, rose hip oil, tea extracts, co-enzyme Q10, collagen, elastin, plant extracts, soy isoflavones, etc. Other suitable ingredients that may be used in the sunscreen formulation include, but are not limited to, ethyl cellulose, polyvinyl alcohol, carboxymethyl cellulose, vegetable gums and clays, proteins and polypeptides, and an alkaline agent such as sodium hydroxide or potassium hydroxide to neutralize, if desired, part of the fatty acids or thickeners which may be present.

According to another embodiment, the present invention features a sunscreen formulation comprising any of the sunscreen compositions described herein, and a pharmaceutically-acceptable sunscreen carrier. Preferably, the sunscreen composition is present in an amount effective to absorb UV radiation. The sunscreen formulation contain pharmaceutically-acceptable sunscreen carriers selected as appropriate for the formulation desired. For example, it is possible to prepare sunscreen formulation of the present invention in the form of organic solvent solutions, aqueous emulsions, gels, or aerosol formulation.

As used herein, the term “cosmeceutically-acceptable sunscreen carrier” or “pharmaceutically-acceptable sunscreen carrier”, means one or more substantially non-irritating compatible filler diluents which are suitable for topical application to the skin of a mammal, i.e. human. The term “compatible”, as used herein, means that the components of the carrier must be capable of being comingled with the sunscreen composition, and with each other, in a manner such that there is no interaction which would substantially reduce the efficacy of the composition during use for protecting the skin from the harmful effects of UV radiation. Cosmeceutically-acceptable and pharmaceutically-acceptable sunscreen carriers must, of course, be of sufficiently high purity and sufficiently low toxicity to render them suitable for topical administration to the mammal. The sunscreen carriers useful in the formulation of the present invention include, for example, water, oils, fats, waxes, synthetic polymers, emulsifiers, surfactants, perfumes, dyes, and preservatives.

In another embodiment, the present invention features a method of protecting a skin of a mammal from harmful photo-biological effects of UV radiation. The method may comprise topically applying to the skin of the mammal an effective amount of any of the sunscreen formulations described herein. Without wishing to limit the invention to a particular theory or mechanism, the UV absorbing compound is incapable of leeching into the skin due to the fact that it is covalently bound to the silane coupling agents.

### Examples

The following are non-limiting examples of the present invention, in particular, the preparation of alkoxysilane modified curcumin monomers and their polymerization to form sunscreen nanoparticles. The examples are for illustrative purposes only and are not intended to limit the invention in any way. Equivalents or substitutes are within the scope of the invention.

Preparation of Curcumin Monomer Modified with Three Triethoxysilyl Groups:

To a 1000 mL round bottom flask, 36.8 grams (0.1 moles) curcumin were dissolved in anhydrous tetrahydrofuran (500 mL). To this excess potassium carbonate (0.5 moles) was added to deprotonate the phenolic and enol hydroxy groups yielding a dark red solution. Then 4 equivalents of glycidyloxypropyltriethoxysilane is added and the resulting solution is refluxed under dry nitrogen for 24 hours. At this point, the reaction is cooled, anhydrous magnesium sulfate is added, and the mixture is filtered through a fritted glass filter to remove residual potassium carbonate. The tetrahydrofuran is removed using roto-evaporation under reduced pressure and the glycidyloxypropyltriethoxysilane is distilled off at 70° C. at 215 microns vacuum. The heavy red oil residue can be obtained in over 50% yield and the degree of modification verified using H Nuclear magnetic resonance spectroscopy to compare the integration of the 27 ethoxy methyl hydrogens at 1.8 ppm and the 18 ethoxy methylene hydrogens at 3.72 ppm with the alkenyl and aromatic resonances between 5-11 ppm. The trisilylated monomer also has a mass spectrometric parent peak at m/z 1202.59.

Preparation of Silica/Silsesquioxane Sunscreen Particles:

Sunscreen particles can be prepared by copolymerization of the curcumin monomer with tetraethoxysilane in ethanol. A 100 mL round-bottom flask was equipped with a thermometer and magnetic stir bar. The flask was charged with anhydrous ethanol (33.1 mL) and aq. NH4OH (5.154 M, 20.1 mL) and the solution stirred at 30° C. In a scintillation vial in the same water bath, a previously prepared 10 mole % precursor solution in TEOS (5 mL) was heated to 30° C. Once the temperatures of both solutions had equilibrated at 30° C., precursor solution was added to the reaction flask and the resultant solution was allowed to stir 15 s. Afterwards, stirring was stopped and the reaction mixture was allowed to stand at 30° C. for 2 hours. Sphere evolution was indicated by an increasing opalescence of the mixture beginning 1-5 minutes after adding the precursor solution. The transition to a turbid white suspension occurred within a few more minutes. After 2 hours, the product was centrifuged to remove excess catalyst and starting material, then washed 3× with ethanol.

Preparation of Silsesquioxane Sunscreen Particles:

A 1000 mL round bottom flask was charged with anhydrous ethanol (500 mL mL) and aq. NH4OH (5.154 M, 40 mL) and the solution stirred at 30° C. The monomer (27.2 grams, 0.0225 moles) in 30 mL ethanol was added to the reaction flask and the resultant solution was allowed to stir 15 s. Afterwards, stirring was stopped and the reaction mixture was allowed to stand at 30° C. for 2 hours. As before particle formation is heralded by increased scattering that lightens the red color of the solution to orange. After 2 hours, the product was filtered to remove excess catalyst and starting material, and the particles were washed 3× with ethanol.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A composition for producing a UV absorbing sunscreen monomer having one or more alkoxysilyl functional groups, comprising:
a. a UV absorbing compound, wherein the UV absorbing compound contains at least one nucleophilic functional group; and
b. one or more silane coupling agents having at least one epoxide group;
wherein the nucleophilic functional group is configured to react with the epoxide group to form a covalent bond, thereby producing the sunscreen monomer, wherein the covalent bond is effective for enhancing photostability of the sunscreen monomer and preventing leeching of the UV absorbing compound from the sunscreen monomer.

2. The composition of claim 1, wherein the nucleophilic functional group of the UV absorbing compound is a hydroxyl moiety, an amine moiety, a thiol moiety, or a carboxylic acid moiety.

3. The composition of claim 2, wherein the nucleophilic functional group is a hydroxyl moiety and the UV absorbing compound is selected from a group consisting of curcumin, resveratrol, catechin, ellagic acid, usnic acid, hematoxylin/hematein, kermesic acid, carminic acid, caffeic acid, ferulic acid, 3,5-dicaffeoylquininic acid, 5,6,7-trihydroxy-2-phenyl-4H-1-benzo-pyran-4-one, salvianolic acid, 2′,4,4′-trihydroxychalcone, 3,5,7,3′,4′,5′-hexa-hydroxyflavone, 3,5,7,3′,4′,5′-hexahydroxyflavone, 3′,4′,5,7-tetrahydroxyflavone, 5,7,4′-trihydroxyflavonol, 5,7-dihydroxy-2-phenylchromen-4-one, 5,7-dihydroxy-3-(4-methoxyphenyl)-4H-1-benzopyran-4-one, 5,7-dihydroxy-2-(4-hydroxyphenyl)-4H-chromen-4-one, 5,7-dihydroxy-3-(4-hydroxyphenyl)-4H-chromen-4-one, and bis-ethylhexyl-oxyphenol methoxyphenyl triazine.

4. The composition of claim 2, wherein the nucleophilic functional group is an amine moiety and the UV absorbing compound is selected from a group consisting of 1,3-phenylenebis((3-aminophenyl)-methanone), 4,4′-diamino-2,2′-stilbenedisulfonic acid, and 2-(4-aminophenyl)-1H-benzimidazole-5-amine).

5. The composition of claim 2, wherein the nucleophilic functional group is a thiol moiety and the UV absorbing compound is selected from a group consisting of thiolated avobenzones, 1,3-bis(4-mercaptophenyl)propane-1,3-dione, oxybenzones, bis(4-mercaptophenyl)-methanone, thiolated coumarins, 7-mercaptocoumarin, cinnamates, 2-ethylhexyl (E)-3-(4-mercaptophenyl)acrylate, salicylates, sec-butyl 2-mercaptobenzoate, anthranilates, 2-mercaptoethyl 2-(methylamino)benzoates, bisoctrizoles, 6,6′-methylenebis(2-(2H-benzo[d][1,2,3]triazol-2-yl)-4-(2,4,4-trimethylpentan-2-yl)-benzenethiol), bemotrizinols, and 6,6′-(6-(4-mercaptophenyl)-1,3,5-triazine-2,4-diyl)bis(3-((2ethylhexyl)oxy)phenol).

6. The composition of claim 2, wherein the nucleophilic functional group is a carboxylic acid moiety and the UV absorbing compound is selected from a group consisting of flavonoids, coumarin-3-carboxylic acid, fumaric acid, carboxylated benzophenones, 4,4′-carbonyldibenzoic acid, 3-(2-(2-(2-carboxyethyl)-6-hydroxybenzoyl)phenyl)propanoic acid, avobenzones, 3-(2-(3-(4-(tert-butyl)-2-(2-carboxyethyl)phenyl)-3-oxopropanoyl)-5-hydroxyphenyl)-propanoic acid, anthranilates, 3-(2-(sec-butoxycarbonyl)-3-(methylamino)-phenyl)propanoic acid, bisoctriazoles, 3,3′-((methylenebis(2-hydroxy-5-(2,4,4-trimethylpentan-2-yl)-3,1-phenylene))bis(2H-benzo[d][1,2,3]triazole-2,4-diyl))-dipropionic acid, bemotrizinols, and 4-(4,6-bis(4-((2-ethylhexyl)oxy)-2-hydroxyphenyl)-1,3,5-triazin-2-yl)benzoic acid.

7. The composition of claim 1, wherein the silane coupling agent is selected from a group consisting of glycidyloxypropyltrimethoxysilane, glycidyloxypropyltriethoxysilane, glycidyloxypropyldimethoxymethylsilane, glycidyloxypropyldiethoxymethylsilane, (2-(7-oxabicyclo[4.1.0]heptan-3-yl)ethyl)-trimethoxysilane, (2-(7-oxabicyclo[4.1.0]heptan-3-yl)ethyl)triethoxysilane, (2-(7-oxabicyclo-[4.1.0]heptan-3-yl)ethyl)dimethoxymethylsilane, (2-(7-oxabicyclo[4.1.0]heptan-3-yl)ethyl)diethoxymethylsilane, trimethoxy(2-(oxiran-2-yl)ethyl)-silane, triethoxy(2-(oxiran-2-yl)ethyl)silane, methyldimethoxy(2-(oxiran-2-yl)ethyl)silane, methyl-diethoxy(2-(oxiran-2-yl)ethyl)silane, trimethoxy(oxiran-2-ylmethyl)silane, triethoxy-(oxiran-2-ylmethyl)silane), methyldimethoxy(oxiran-2-ylmethyl)silane, and methyl-diethoxy(oxiran-2-ylmethyl)silane.

8. A sunscreen nanoparticle formed by polymerization of the alkoxysilyl groups of the sunscreen monomer of claim 1.

9. The sunscreen nanoparticle of claim 8, wherein the polymerization further comprises co-polymerization with silicate comonomers.

10. The sunscreen nanoparticle of claim 9, wherein the silicate comonomers are selected from a group consisting of tetraalkoxysilane comonomers, sodium silicate comonomers, and organotrialkoxysilane comonomers.

11. The sunscreen nanoparticle of claim 10, wherein the tetraalkoxysilane comonomers are selected from a group consisting of tetramethoxysilane comonomers and tetraethoxysilane comonomers.

12. The sunscreen nanoparticle of claim 8, wherein the polymerization is a sol-gel polymerization or an emulsion polymerization.

13. A sunscreen formulation, comprising:
a. the sunscreen nanoparticles of claim 8, wherein the sunscreen nanoparticles are present in an amount effective to absorb UV radiation; and
b. a pharmaceutically-acceptable sunscreen carrier.

14. A method of producing a photochemically stable, non-leeching sunscreen monomer for use in a sunscreen formulation, said method comprising:
a. providing a UV absorbing compound, wherein the UV absorbing compound contains at least one nucleophilic functional group;
b. providing one or more silane coupling agents having at least one epoxide group; and
c. reacting the UV absorbing compound with the one or more silane coupling agents, wherein the nucleophilic functional group of the UV absorbing compound reacts with the epoxide group of the silane coupling agent to form a covalent bond, thereby forming the sunscreen monomer;
wherein the covalent bond between the UV absorbing compound and the silane coupling agent is effective for enhancing photostability of the sunscreen monomer and preventing leeching of the UV absorbing compound from the sunscreen monomer.

15. The method of claim 14, wherein the nucleophilic functional group of the UV absorbing compound is a hydroxyl moiety, an amine moiety, a thiol moiety, or a carboxylic acid moiety.

16. The method of claim 15, wherein the nucleophilic functional group is a hydroxyl moiety and the UV absorbing compound is selected from a group consisting of curcumin, resveratrol, catechin, ellagic acid, usnic acid, hematoxylin/hematein, kermesic acid, carminic acid, caffeic acid, ferulic acid, 3,5-dicaffeoylquininic acid, 5,6,7-trihydroxy-2-phenyl-4H-1-benzopyran-4-one, salvianolic acid, 2′,4,4′-trihydroxychalcone, 3,5,7,3′,4′,5′-hexahydroxyflavone, 3,5,7,3′,4′,5′-hexahydroxyflavone, 3′,4′,5,7-tetrahydroxyflavone, 5,7-dihydroxy-2-phenylchromen-4-one, 5,7-dihydroxy-3-(4-methoxyphenyl)-4H-1-benzopyran-4-one, 5,7,4′-trihydroxyflavonol, 5,7-dihydroxy-2-(4-hydroxyphenyl)-4H-chromen-4-one, 5,7-dihydroxy-3-(4-hydroxyphenyl)-4H-chromen-4-one, and bis-ethylhexyl-oxyphenol methoxyphenyl triazine.

17. The method of claim 15, wherein the nucleophilic functional group is an amine moiety and the UV absorbing compound is selected from a group consisting of 1,3-phenylenebis((3-aminophenyl)methanone),4,4′-diamino-2,2′-stilbenedisulfonic acid, and 2-(4-aminophenyl)-1H-benzimidazole-5-amine).

18. The method of claim 15, wherein the nucleophilic functional group is a thiol moiety and the UV absorbing compound is selected from a group consisting of thiolated avobenzones, 1,3-bis(4-mercaptophenyl)propane-1,3-dione, oxybenzones, bis(4-mercaptophenyl)methanone, thiolated coumarins, 7-mercaptocoumarin, cinnamates, 2-ethylhexyl (E)-3-(4-mercaptophenyl)-acrylate, salicylates, sec-butyl 2-mercaptobenzoate, anthranilates, 2-mercaptoethyl 2-(methylamino)benzoates, bisoctrizoles, 6,6′-methylenebis(2-(2H-benzo[d][1,2,3]triazol-2-yl)-4-(2,4,4-trimethylpentan-2-yl)benzenethiol), bemotrizinols, and 6,6′-(6-(4-mercaptophenyl)-1,3,5-triazine-2,4-diyl)bis(3-((2ethylhexyl)oxy)phenol).

19. The method of claim 15, wherein nucleophilic functional group is a carboxylic acid moiety and the UV absorbing compound is selected from a group consisting of flavonoids, coumarin-3-carboxylic acid, carboxylated benzophenones, 4,4′-carbonyldibenzoic acid, 3-(2-(2-(2-carboxyethyl)-6-hydroxy-benzoyl)phenyl)propanoic acid, fumaric acid, anthranilates, 3-(2-(sec-butoxycarbonyl)-3-(methylamino)phenyl)propanoic acid, avobenzones, 3-(2-(3-(4-(tert-butyl)-2-(2-carboxyethyl)phenyl)-3-oxopropanoyl)-5-hydroxyphenyl)-propanoic acid, bisoctriazoles, 3,3′-((methylenebis(2-hydroxy-5-(2,4,4-trimethylpentan-2-yl)-3,1-phenylene))bis(2H-benzo[d][1,2,3]triazole-2,4-diyl))dipropionic acid, bemotrizinols, and 4-(4,6-bis(4-((2-ethylhexyl)oxy)-2-hydroxyphenyl)-1,3,5-triazin-2-yl)benzoic acid.

20. The method of claim 14, wherein the silane coupling agent is selected from a group consisting of glycidyloxypropyltrimethoxysilane, glycidyloxypropyltriethoxysilane, glycidyloxypropyldimethoxymethylsilane, glycidyloxypropyldiethoxymethylsilane, (2-(7-oxabicyclo[4.1.0]heptan-3-yl)ethyl)trimethoxysilane, (2-(7-oxabicyclo[4.1.0]heptan-3-yl)ethyl)triethoxysilane, (2-(7-oxabicyclo[4.1.0]heptan-3-yl)ethyl)dimethoxymethylsilane, (2-(7-oxabicyclo[4.1.0]heptan-3-yl)ethyl)-diethoxymethylsilane, trimethoxy(2-(oxiran-2-yl)ethyl)silane, triethoxy(2-(oxiran-2-yl)ethyl)silane, methyldimethoxy(2-(oxi ran-2-yl)ethyl)silane, methyldiethoxy(2-(oxiran-2-yl)ethyl)silane, trimethoxy(oxiran-2-ylmethyl)silane, triethoxy(oxiran-2-ylmethyl)silane), methyldimethoxy(oxiran-2-ylmethyl)silane, and methyldiethoxy-(oxiran-2-ylmethyl)silane).
